(19) United States
(12) Patent Application Publication
(10) Pub. No.: US 2026/0210931 A1
(43) Pub. Date: Jul. 23, 2026

(54) APPARATUS AND METHOD FOR DETECTING ABNORMALITIES IN CONTINUOUS CASTING PROCESS

(71) Applicant: Hyundai Steel Company, Incheon (KR)

(72) Inventors: Joon Suk Han, Incheon (KR); Se Ho Lim, Incheon (KR); Hun Gue Lee, Incheon (KR); Hee Chel Chae, Incheon (KR)

(21) Appl. No.: 19/445,982

(22) Filed: Jan. 12, 2026

(30) Foreign Application Priority Data
Jan. 22, 2025 (KR) 10-2025-0009706

(51) Int. Cl. G01N 33/205; B22D 11/20

(52) U.S. Cl. CPC G01N 33/205; B22D 11/201

(57) ABSTRACT

The present disclosure relates to an apparatus for detecting abnormalities in a continuous casting process, the apparatus including: a sensor module configured to detect a mold level and a stopper position in the continuous casting process; and a processor configured to learn information about the mold level and the stopper position detected by the sensor module and determine a mold level hunting occurrence type and a response method.

### CROSS-REFERENCE TO RELATED APPLICATION

This application claims priority from and the benefit of Korean Patent Application No. 10-2025-0009706, filed on Jan. 22, 2025, which is hereby incorporated by reference for all purposes as if set forth herein.

### BACKGROUND

### Field

The present disclosure relates to an apparatus and method for detecting abnormalities in a continuous casting process, which automatically detects declogging and clogging phenomena by analyzing stopper position and mold level data in the continuous casting process, and based on this, provides operation guidelines.

### Discussion of the Background

In general, the continuous casting process is a process of producing a semi-finished slab by injecting high-temperature molten metal (i.e., molten steel) into a mold, and is mainly used to produce high-quality steel products such as automobile steel plates and construction steel.

The surface condition of the molten steel in the mold (i.e., the condition of the melt surface) directly affects the slab quality, and the surface stability of the molten steel in the mold (i.e., the stability of the mold level) is very important for preventing surface defects of the product and forming a uniform solidification layer.

To this end, the existing continuous casting process mainly used a method of monitoring the mold level and stopper position depending on the experience of the operator and manually adjusting the operation if necessary.

In the past, there was a problem in that an operator manually detected and responded to abnormal changes in mold level and stopper position, resulting in a lack of consistency in abnormality judgment and causing differences in product quality depending on the response measures taken based on the operator's skill level.

Furthermore, in the past, when phenomena such as declogging and clogging occurred, there was no system to quickly and accurately detect them, which could lead to unnecessary adjustments during the continuous casting process or a decline in product quality.

In addition, in the past, there was a problem in that it took time to solve the problem due to the lack of quantitative data to systematically analyze the cause of surface defects in products caused by mold level instability.

In addition, in the past, when molten steel flow instability due to the clogging phenomenon occurred, it was not clear how to immediately solve the issue, resulting in the problems of operational delays and additional costs. Furthermore, since an automated analysis and response system utilizing mold level and stopper position data was not sufficiently developed, there was a limit to the improvement of continuous casting process efficiency and quality.

The background technology of the present disclosure is disclosed in Korean Patent Laid-Open Publication No. 10-2017-0068645 (published on Jun. 20, 2017, Cast Manufacturing Method).

The above-described information disclosed in the Background of the Invention is intended solely to enhance the understanding of the background of the present disclosure, and may thus include information that does not constitute the prior art.

### SUMMARY

According to one aspect of the present disclosure, the present disclosure provides an apparatus and method for detecting abnormalities in a continuous casting process, which automatically detects declogging and clogging phenomena by analyzing stopper position and mold level data in the continuous casting process, and based on this, provides operation guidelines.

However, the technical problems to be solved by the present disclosure are not limited to the above-mentioned problems, and other problems not mentioned will be clearly understood by those skilled in the art from the description of the disclosure presented below.

An apparatus for detecting abnormalities in a continuous casting process according to one aspect of the present disclosure includes: a sensor module configured to detect a mold level and a stopper position in the continuous casting process; and a processor configured to learn information about the mold level and the stopper position detected by the sensor module and determine a mold level hunting occurrence type and a response method.

In the present disclosure, the processor, for the purpose of the learning, may collect the mold level and stopper position information in real time, analyze time-series changes in the data using a convolutional neural network (CNN) model, and extract features that appear when declogging occurs.

In the present disclosure, the processor may apply the features extracted through the CNN model to a support vector data description (SVDD) technique to learn the distribution of normal and abnormal data, and determine abnormal data falling outside the normal range as declogging.

In the present disclosure, the processor may collect the mold level and stopper position information and apply a cumulative sum control chart (CUSUM) model to determine the presence or absence of clogging.

In the present disclosure, the processor, based on the CUSUM model, may accumulate the instantaneous change amount of the stopper position to detect real-time abnormal state, calculate the change amount of the stopper position in the short term to evaluate the degree of blockage over a short period of time, and analyze the overall clogging progression state by checking whether the blockage state is accumulating in the long term.

In the present disclosure, the processor, based on the CUSUM model, may comprehensively learn the features of gradual instability of the mold level, continuous rising pattern of the stopper position, and data showing outliers in the CUSUM indicators when clogging occurs, thereby detecting the presence or absence of clogging in real time.

A method for detecting abnormalities in a continuous casting process according to another aspect of the present disclosure includes: detecting a mold level and a stopper position in the continuous casting process through a sensor module; and allowing a processor to learn information about the mold level and the stopper position detected by the sensor module and determine a mold level hunting occurrence type and a response method.

In the present disclosure, in the determining of the mold level hunting occurrence type and the response method, the processor, for the purpose of the learning, may collect the mold level and stopper position information in real time, analyze time-series changes in the data using a convolutional neural network (CNN) model, and extract features that appear when declogging occurs.

In the present disclosure, in the determining of the mold level hunting occurrence type and the response method, the processor may apply the features extracted through the CNN model to a support vector data description (SVDD) technique to learn the distribution of normal and abnormal data, and determine abnormal data falling outside the normal range as declogging.

In the present disclosure, in the determining of the mold level hunting occurrence type and the response method, the processor may collect the mold level and stopper position information and apply a cumulative sum control chart (CUSUM) model to determine the presence or absence of clogging.

In the present disclosure, in the determining of the mold level hunting occurrence type and the response method, the processor, based on the CUSUM model, may accumulate the instantaneous change amount of the stopper position to detect real-time abnormal state, calculate the change amount of the stopper position in the short term to evaluate the degree of blockage over a short period of time, and analyze the overall clogging progression state by checking whether the blockage state is accumulating in the long term.

In the present disclosure, in the determining of the mold level hunting occurrence type and the response method, the processor, based on the CUSUM model, may comprehensively learn the features of gradual instability of the mold level, continuous rising pattern of the stopper position, and data showing outliers in the CUSUM indicators when clogging occurs, thereby detecting the presence or absence of clogging in real time.

According to one aspect of the present disclosure, the present disclosure automatically detects declogging and clogging phenomena based on stopper position and mold level data in a continuous casting process, thereby having the effect of reducing errors caused by an operator's subjective judgment and ensuring process consistency.

The present disclosure automatically provides appropriate operating instructions according to declogging and clogging states, thereby having the effect of shortening continuous casting process downtime and minimizing the occurrence of product surface defects caused by mold level instability.

The present disclosure has the effect of improving operating efficiency through data-based quantitative analysis and automation, and consequently, reducing production costs and improving product quality at the same time.

However, the effects achieved through the present disclosure are not limited to the aforementioned effects, and other technical effects not mentioned will be clearly understood by those skilled in the art from the description of the disclosure presented below.

### BRIEF DESCRIPTION OF THE DRAWINGS

FIG. 1 is an exemplary diagram showing a schematic configuration of an apparatus for detecting abnormalities in a continuous casting process according to one embodiment of the present disclosure.

FIG. 2 is an exemplary diagram showing a schematic configuration of a continuous casting apparatus.

FIG. 3 is an exemplary diagram schematically illustrating a cross-sectional configuration of a mold in FIG. 2 in order to describe a mold level.

FIG. 4 is an exemplary diagram for illustrating characteristics of a declogging phenomenon and a clogging phenomenon as a cause of mold level hunting.

FIG. 5 is a flowchart for illustrating a learning method for determining a declogging phenomenon according to one embodiment of the present disclosure.

FIG. 6 is a flowchart for illustrating a learning method for determining a clogging phenomenon according to one embodiment of the present disclosure.

FIG. 7 is an exemplary graph showing test results obtained using an apparatus for detecting abnormalities in a continuous casting process according to one embodiment of the present disclosure.

### DETAILED DESCRIPTION OF THE ILLUSTRATED EMBODIMENTS

Hereinafter, an embodiment of the present disclosure will be described with reference to the attached drawings.

In this process, the thickness of lines, sizes of components, and the like illustrated in the drawings may be exaggerated for clarity and convenience of explanation. Furthermore, the terms described below are terms defined in consideration of their functions in the present disclosure, and may vary depending on the intentions or practices of a user or operator. Therefore, the definitions of these terms should be based on the content throughout this specification.

Hereinbelow, with reference to the accompanying drawings, embodiments of the present disclosure will be described in detail so as to be easily implemented by one of ordinary skill in the art to which the present disclosure pertains. However, the present disclosure may be embodied in a variety of forms and is not limited to the embodiments described herein. In addition, in order to clearly describe the present disclosure in the drawing, parts irrelevant to the description are omitted from the drawings, and similar reference numerals are used for similar parts throughout the specification.

Throughout the specification, when a part is described as “including/comprising” a certain component, it means that other components may be further included/comprised, rather than excluded, unless otherwise stated.

The terms or words used in the specification and claims of the present application should not be construed as being limited to their ordinary or dictionary meanings, but should be interpreted as meanings and concepts consistent with the technical spirit of the present disclosure, based on the principle that the inventor may adequately define the concepts of terms to best describe his/her disclosure.

Therefore, it should be understood that the embodiments described in this specification and the configurations shown in the drawings are only some of the most preferred embodiments of the present disclosure and do not represent the entire technical concept of the present disclosure, and thus, various equivalents and modifications may be substituted for them at the time of filing this application. Furthermore, as used herein, the terms “comprise/include” and/or “comprising/including” specify the presence of the mentioned shapes, numbers, steps, operations, members, elements, and/or groups thereof, and do not exclude the presence or addition of one or more other shapes, numbers, steps, operations, members, elements, and/or groups thereof. In addition, when describing embodiments of the present disclosure, the terms “may” and “may be” mean that “one or more embodiments of the present disclosure” may be included.

In addition, in order to aid in understanding the disclosure, the accompanying drawings may not be drawn to scale, and the dimensions of some components may be exaggerated. In addition, the same reference numerals may be assigned to the same components in different embodiments.

Referring to two compared objects as being “identical” means “substantially identical.” Accordingly, substantial identity may include deviations that are considered low in the art, for example, within 5%. In addition, uniformity of a parameter over a given region may mean uniformity on average.

Although the terms ‘first’, ‘second’, etc. are used to describe various components, it goes without saying that these components are not limited by these terms. These terms are only used to distinguish one component from another, and of course, a first component may also be a second component unless otherwise specifically stated.

Throughout the specification, unless otherwise specifically stated, each component may be singular or plural.

When any component is disposed “on (or under)” a component or “above (or below)” a component below, this may mean not only that any component is disposed in contact with the upper surface (or lower surface) of said component, but also that other components may be interposed between said component and any component disposed on (or under) said component.

In addition, when it is described that a component is “connected”, “coupled” or “joined” to another component, it should be understood that the components may be directly connected or joined to each other, but that other components may be “interposed” between the respective components, or that the respective components may be “connected”, “coupled” or “joined” through another component. In addition, when it is said that a part is “electrically coupled” to another part, this includes not only cases where they are directly coupled, but also cases where they are indirectly coupled with another element interposed therebetween.

Throughout the specification, the expression “A and/or B” refers to A, B, or A and B, unless otherwise specifically stated. In other words, “and/or” includes any or all combinations of the plurality of items listed. The expression “C to D” means C or more and D or less, unless otherwise specifically stated.

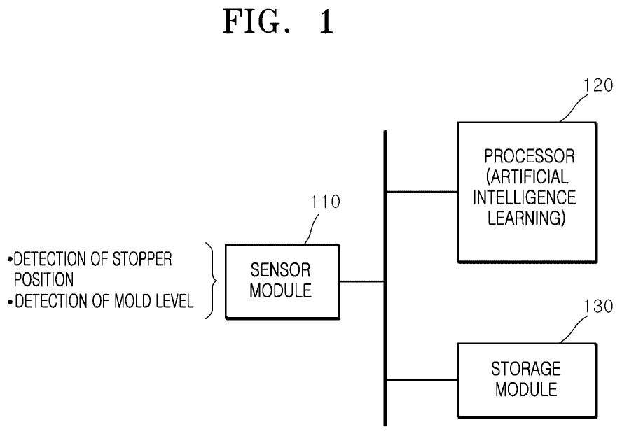

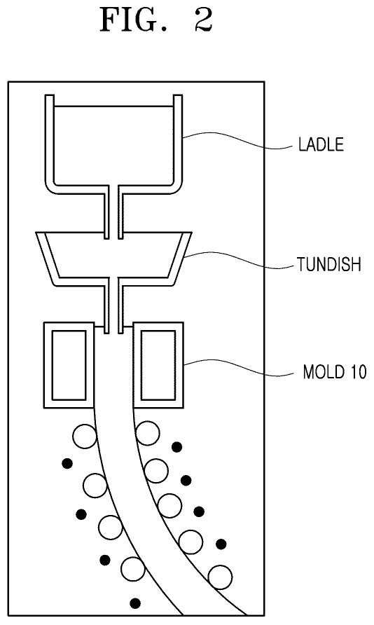

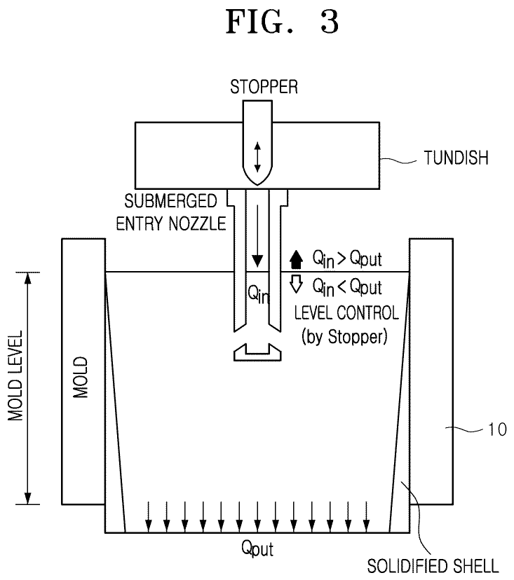

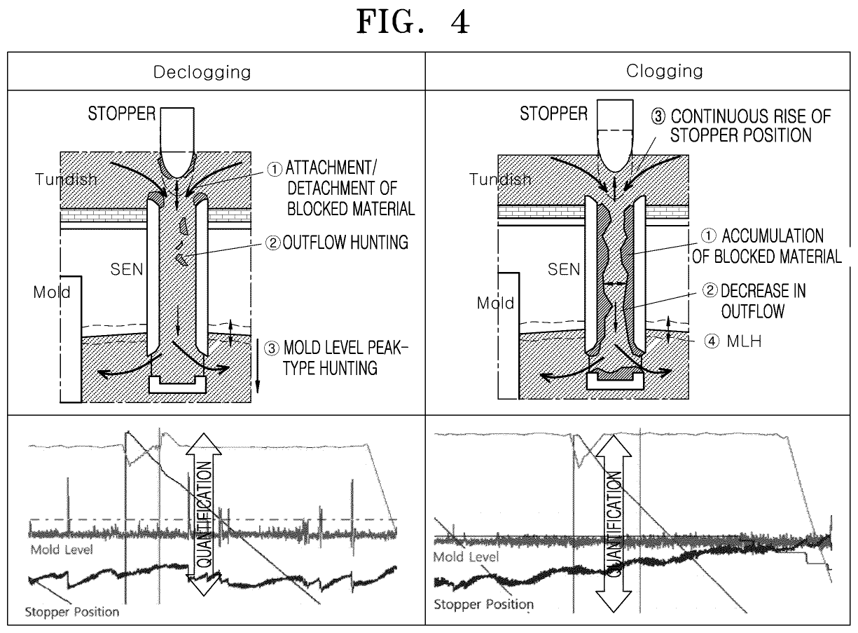

FIG. 1 is an exemplary diagram showing a schematic configuration of an apparatus for detecting abnormalities in a continuous casting process according to one embodiment of the present disclosure, FIG. 2 is an exemplary diagram showing a schematic configuration of a continuous casting apparatus, and FIG. 3 is an exemplary diagram schematically illustrating a cross-sectional configuration of a mold in FIG. 2 in order to describe a mold level. FIG. 4 is an exemplary diagram for illustrating characteristics of a declogging phenomenon and a clogging phenomenon as a cause of mold level hunting.

Referring to FIG. 1, an apparatus for detecting abnormalities in a continuous casting process according to the present embodiment may include a sensor module 110, a processor 120, and a storage module 130.

The sensor module 110 may include a sensor for measuring a height of a mold melt surface (i.e., mold level). Here, the sensor for measuring a height of a mold melt surface may be a non-contact sensor, such as a radiation sensor or an eddy current sensor.

The sensor module 110 may include a sensor for detecting a stopper position. Here, the stopper position may be estimated based on a control signal of an actuator (mainly a servo motor or hydraulic actuator) that drives a stopper.

In the present embodiment, the sensor module 110 is a concept that includes a sensor, a sensor driving circuit, an actuator, and an actuator driving circuit.

The sensor module 110 may be installed vertically above the mold melt surface, installed inside the mold at a similar height to the mold melt surface, or installed at the upper edge of the mold.

Referring to FIG. 3, the mold level refers to a height from the bottom of the mold copper plate to the mold melt surface, and stable mold level control is required to maintain consistent product quality.

Here, the mold level control refers to maintaining the mold level (or melt surface height) constant by adjusting the rate of molten steel injected into the mold 10 according to the casting speed.

For example, when the molten steel injection rate (Qᵢₙ) is excessive compared to the molten steel discharge rate (Qₒᵤₜ) in the mold 10, the mold level (or melt surface height) rises. Conversely, when the molten steel discharge rate (Qₒᵤₜ) is excessive compared to the molten steel injection rate (Qᵢₙ) in the mold 10, the mold level (or melt surface height) falls. Therefore, the mold level control is performed to maintain the mold level (or melt surface height) constant by adjusting the molten steel injection rate in the mold 10.

For reference, referring to FIGS. 2 and 3, molten steel is injected from a ladle into a tundish, and the molten steel discharged from the tundish is injected into the mold 10 through a submerged entry nozzle (SEN), wherein the molten steel flowing into the SEN is controlled by a stopper above the SEN.

The sensor module 110 may measure (or detect) the mold level (or melt surface height) and stopper position at specified time intervals (e.g., 1 s, 10 s, etc.).

The processor 120 corresponds to a controller, and may accumulate and store the mold level (or melt surface height) and stopper position measured (or detected) by the sensor module 110 in the storage module 130.

The processor 120 may learn (e.g., machine learning, deep learning, etc.) the mold level (or melt surface height) and stopper position information measured (or detected) by the sensor module 110, and based on the learning results, may determine a mold level hunting occurrence type and a response method.

Here, the mold level hunting refers to a phenomenon in which the molten metal (i.e., molten steel) level within the mold 10 is not constant and fluctuates periodically or aperiodically. When the mold level hunting phenomenon occurs, material blocked by the submerged entry nozzle is incorporated into the molten steel, which may lead to a deterioration in the quality of products (e.g., steel plates) produced in the continuous casting process, and a risk of surface or internal defects occurring in the product.

There are various causes of mold level hunting in the continuous casting process.

For example, as shown in FIG. 4, the causes of mold level hunting include: a declogging phenomenon in which a material blocked in a nozzle falls off or is removed, suddenly making the flow smooth, and a clogging phenomenon in which non-metallic inclusions, slag, alumina, or other impurities gradually accumulate inside the submerged entry nozzle (SEN), gradually reducing or blocking the flow of molten iron.

The declogging may occur when non-metallic inclusions (e.g., alumina inclusions) or slag accumulated inside the submerged entry nozzle (SEN) suddenly fall off due to an increase in the flow velocity of the molten steel, vibration, or thermal changes. In other words, the declogging is a phenomenon that occurs when non-metallic inclusions or slag accumulated inside the submerged entry nozzle suddenly fall off due to an increase in the flow rate of the molten steel, vibration, or thermal changes. When the declogging occurs, the flow rate of molten steel increases rapidly, the mold level temporarily fluctuates greatly, and the stopper position changes rapidly. When the declogging phenomenon occurs in this way, the flow of molten steel suddenly becomes unstable, causing surface ripples in the mold, and non-metallic inclusions or slag flow into the mold, increasing the possibility of defects occurring on the surface of the slab product.

Meanwhile, the clogging occurs due to chemical reactions between an alumina in the molten steel and the inner wall of the submerged entry nozzle or slag, attachment of solidified molten steel or non-metallic substances due to temperature differences between the inside and outside of the submerged entry nozzle, or attachment of inclusions when the molten steel forms turbulence or changes in speed inside the submerged entry nozzle. In other words, the clogging is a phenomenon that occurs due to chemical reactions between an alumina in the molten steel and the inner wall of the submerged entry nozzle or slag, attachment of solidified molten steel or non-metallic substances due to temperature differences between the inside and outside of the submerged entry nozzle, or attachment of inclusions when the molten steel forms turbulence or changes in speed inside the submerged entry nozzle. When the clogging occurs, the molten steel flow gradually decreases, mold level variability increases, and the stopper position gradually rises. When the clogging occurs in this way, there is a problem in that the unstable flow of the molten steel makes uniform solidification in the mold difficult, and nozzle replacement or cleaning operations is required to incur additional time and costs.

In this case, conventionally, there was a problem in that as an operator manually detected and responded to abnormal changes in the mold level and stopper position, there was a lack of consistency in judging the type of abnormality, and the judgment of the type of abnormality occurrence and the response according to the judgment were different depending on the skill level of the operator, thereby resulting in differences in the quality of products (e.g., slabs).

Accordingly, the present embodiment may learn the mold level (or melt surface height) and stopper position information (e.g., machine learning, deep learning, etc.), and based on the learning results, may present the mold level hunting (or mold level abnormality) occurrence types and response methods to the operator (user).

The processor 120 may be implemented to automatically detect a declogging occurrence pattern by learning the pattern based on the mold level and stopper position data as a learning method for determining the declogging phenomenon. However, depending on the embodiments, the functions of the processor 120 may be implemented to be performed using a server (not shown) including at least one processor 120.

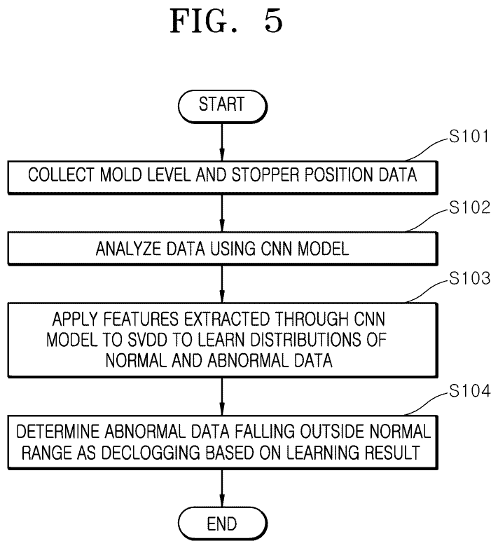

FIG. 5 is a flowchart for illustrating a learning method for determining a declogging phenomenon according to one embodiment of the present disclosure.

Referring to FIG. 5, the processor 120 collects the mold level and stopper position information (or data) in real time (S101), analyzes the time-series changes in the data using a convolutional neural network (CNN) model, and extracts features (or feature vectors) that appear when declogging occurs (S102).

Thereafter, the processor 120 applies (inputs) the features (or feature vectors) extracted through the CNN model to a support vector data description (SVDD) technique to learn the distribution of normal and abnormal data (S103), and through this, determines abnormal data falling outside the normal range as declogging (S104).

For reference, the SVDD distinguishes between normal and abnormal data among the feature data extracted by the CNN, sets a feature space of the normal data, and checks whether data falls outside the normal range during declogging. In other words, it is determined whether or not declogging has occurred based on the distribution of normal state data.

Here, the learning process is performed by distinguishing between labeled normal data and declogging data, and utilizes the rapid fluctuations in mold level and the simultaneous changes in stopper position during declogging as the primary learning target patterns. Finally, the trained CNN+SVDD model enables real-time detection and accurate judgment of declogging phenomena, improving operational efficiency and quality stability.

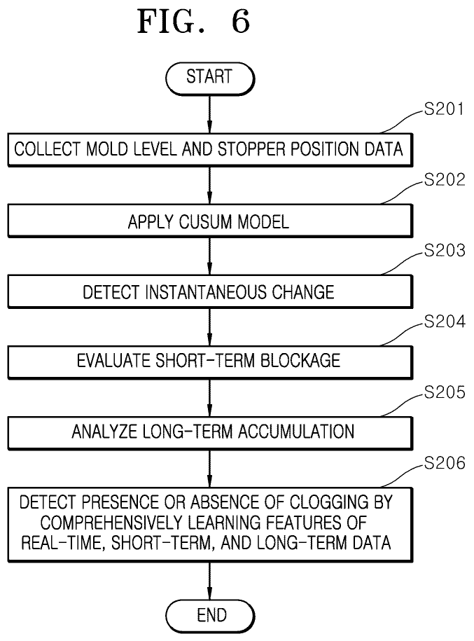

FIG. 6 is a flowchart for illustrating a learning method for determining a clogging phenomenon according to one embodiment of the present disclosure.

Referring to FIG. 6, the processor 120 collects real-time data on mold level and stopper position (S201) and applies a cumulative sum control chart (CUSUM) model (S202).

Here, the CUSUM is primarily used to detect changes in the average value of a process, and is a model that detects anomalies occurring over a certain period of time through the accumulated sum of abnormal data. That is, the CUSUM is a model that detects abnormal changes in data by accumulating differences between the reference value set at the beginning of operation and the current measured value. This allows for quantitative analysis of the gradual changes and patterns of the mold level and stopper position when clogging occurs.

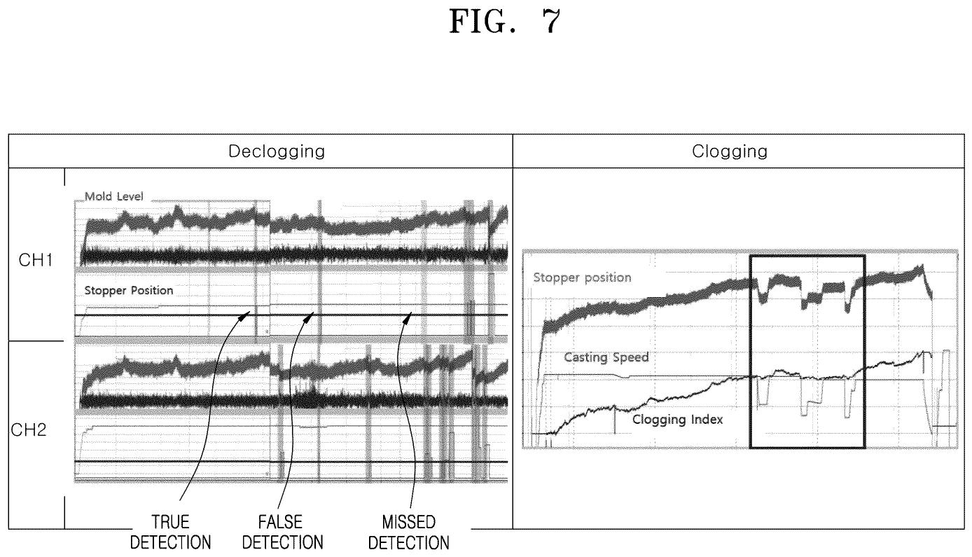

For reference, the above-described CNN+SVDD model is suitable for learning large-scale data, but it may be inefficient to train all data as it is. Accordingly, when data is preprocessed by applying the CUSUM, only data suspected of abnormalities (i.e., data in which the stopper position fluctuates from the average pattern due to the influence of the casting speed, as shown in the boxed region of the clogging graph in FIG. 7) may be extracted and excluded, thereby increasing computational efficiency and improving performance.

Hereinafter, a more detailed description will be provided to facilitate understanding of the CUSUM model applied in the present embodiment. However, the mathematical equations described below are illustrative and not intended to be limiting.

In order to detect a clogging phenomenon, Mathematical Equation 1 compares changes in stopper position with a reference value to calculate the accumulated change.

$${C}_{l} = {\sum }_{t} ( {O}_{t} - {O}_{s 1} ) + {\sum }_{t} ( {O}_{t} - {O}_{s 3} ) + … \qquad [ Mathematical Equation 1 ]$$

Cₗ=Y: Total sum of stopper position fluctuations
Oₜ: Stopper position at a specific point in time (t)
Oₛₙ: Stopper position serving as a reference when operating conditions change (e.g., Oₛ₁, Oₛ₃)

This calculates how much the stopper position deviates from the reference value and accumulates this value to detect clogging. That is, the CUSUM accumulates how much the stopper position has changed at every moment to show the ‘degree of deviation from the normal state’. For example, if the stopper position steadily increases, the Cₗ value gradually increases, indicating the possibility that clogging has occurred. In this case, the unit time may be calculated in units of 1 second, and real-time detection is possible.

Mathematical Equation 2 is an equation representing the degree of clogging (i.e., the degree of blockage) Y′, and can evaluate the degree of clogging by calculating the amount of change in the stopper position over a short period of time (e.g., 10 seconds).

$${Y}^{′} ( 11 s ) = Y ( 11 ) - Y ( 1 ) \qquad [ Mathematical Equation 2 ]$$

Y(11): Stopper position at 11 seconds
Y(1): Stopper position at 1 second

That is, it indicates how much the stopper position has changed from 1 second to 11 seconds. That is, if the stopper moves significantly over a period of 10 seconds, the Y′ value increases, which means that the blockage is becoming more severe. Conversely, if there is little change in the stopper position, the Y′ value is small. It is calculated in units of 10 seconds.

Mathematical Equation 3 is an equation that calculates C′ₗ representing the degree of blockage accumulation, may evaluate whether the blockage state is accumulating over the long term, and may determine the progression degree of the clogging state by reflecting both past and present data.

$${C}_{l}^{′} = {\sum }_{i = 0}^{i - 1} {Y}_{i}^{′} + {Y}^{′} \qquad [ Mathematical Equation 3 ]$$

$${Y}_{i}^{′} :$$

Blockage degree (Y′) calculated at each time interval

The cumulative sum is calculated to indicate whether the clogging is worsening over time. In other words, the progression degree of the clogging is assessed by continuously adding the Y′ values. For example, if the stopper position continues to change slightly every moment, the C′ₗ value becomes larger, indicating that the clogging is intensifying. In this case, the unit time is calculated per second or at each operational change point.

In summary, the real-time abnormal state may be detected by accumulating the instantaneous change amount of the stopper position through Cₗ (S203), the degree of blockage over a short period of time may be evaluated by calculating the change amount of the stopper position in the short term through Y′ (S204), and the overall clogging progression state may be analyzed by checking whether the blockage state is accumulating in the long term through

$${C}_{l}^{′}$$

### (S205).

For example, assuming that the stopper position rises rapidly for 10 seconds and the clogging is occurring, Cₗ detects the possibility of clogging as the cumulative change amount per second gradually increases compared to the reference value; if the stopper position has changed significantly for 10 seconds, the Y′ value increases, indicating that the degree of clogging is severe; and

$${C}_{l}^{′}$$

may indicate that the clogging is gradually intensifying in the long term by accumulating clogging data for the previous 10 seconds and comparing it to the current situation.

As described above, the present embodiment can analyze the clogging state by utilizing all of real-time, short-term (10 seconds) and long-term data.

Finally, the learned model comprehensively learns the features of gradual instability of the mold level, continuous rising pattern of the stopper position, and data showing outliers in the CUSUM indicators when clogging occurs, and based on this, accurately detects the presence or absence of clogging in real time (S206).

As described above, the present embodiment can accurately detect the cause of the abnormality and accordingly provide consistent response methods (e.g., operation guidelines) to the operator, thereby significantly improving operation efficiency and product quality without relying on the operator's judgment.

FIG. 7 is an exemplary graph showing test results obtained using an apparatus for detecting abnormalities in a continuous casting process according to one embodiment of the present disclosure.

Referring to FIG. 7, Channel 1 and Channel 2 represent the test results for two submerged entry nozzles, respectively, during declogging. Channel 1 and Channel 2 represent data collected from different channels, and show the results of comparing and analyzing data measured from different points in time or perspectives for the same phenomenon.

When a declogging phenomenon occurs, the mold level temporarily fluctuates rapidly, and a pattern of rapid rises and falls in the mold level appears. Furthermore, when a declogging phenomenon occurs, a pattern in which the stopper position fluctuates rapidly or a control action occurs appears.

High consistency results could be obtained with about 87.3% in the case of a true detection operation that accurately detects the point where declogging actually occurred, about 3.7% in the case of a false detection operation that incorrectly detects declogging even though it did not occur, and about 9% in the case of a missed detection operation that fails to detect declogging even though it actually occurred.

Referring again to FIG. 7, a pattern in which the stopper position gradually increases when the cloaking occurs is shown. This is because, as the flow of molten steel becomes blocked, the stopper gradually rises to compensate for it.

The clogging index represents data that quantifies the degree of clogging based on CUSUM or another formula, and affects the stopper position according to the casting speed, but exhibits a pattern of steadily increasing beyond a certain threshold when clogging occurs.

As described above, in the present embodiment, the processor 120 can determine the declogging phenomenon and the clogging phenomenon through machine learning, thereby supporting the operator to detect and respond to the problem in real time.

As shown in the graph in FIG. 7, the present embodiment may utilize major patterns (mold level, stopper position, clogging index, etc.) in declogging and clogging states as learning data to detect abnormal situations and determine response methods through a machine learning model (CNN+SVDD, etc.), thereby significantly improving operation efficiency and quality stability.

As described above, the present embodiment automatically detects declogging and clogging phenomena based on stopper position and mold level data in the continuous casting process, thereby having the effect of reducing errors caused by an operator's subjective judgment and ensuring process consistency. Furthermore, the present embodiment automatically provides appropriate operating instructions according to declogging and clogging states, thereby having the effect of shortening process downtime and minimizing the occurrence of product surface defects caused by mold level instability. In addition, the present embodiment has the effect of improving operating efficiency through data-based quantitative analysis and automation, and consequently, reducing production costs and improving product quality at the same time.

Although the present disclosure has been described above with reference to the embodiments shown in the drawings, these are merely exemplary, and those of ordinary skill in the art will understand that various modifications and other equivalent embodiments are possible from the above. Accordingly, the technical protection scope of the present disclosure should be defined by the following claims. Furthermore, the implementations described herein may be implemented, for example, as methods or processes, devices, software programs, data streams, or signals. Even if discussed only in the context of a single form of implementation (e.g., only as a method), the implementation of the discussed features may also be implemented in other forms (e.g., as a device or program). The devices may be implemented using appropriate hardware, software, firmware, etc. The methods may be implemented in a device such as a processor, which generally refers to a processing device including, for example, a computer, a microprocessor, an integrated circuit, or a programmable logic device. The processor also includes communication devices such as computers, cell phones, personal digital assistants (PDAs), and other devices that facilitate the communication of information between end users.

What is claimed is:

1. An apparatus for detecting abnormalities in a continuous casting process, the apparatus comprising:
a sensor module configured to detect a mold level and a stopper position in the continuous casting process; and
a processor configured to learn information about the mold level and the stopper position detected by the sensor module and determine a mold level hunting occurrence type and a response method.

2. The apparatus of claim 1, wherein the processor:
collects the mold level and stopper position information in real time,
analyzes time-series changes in the data using a convolutional neural network (CNN) model, and
extracts features that appear when declogging occurs.

3. The apparatus of claim 1, wherein the processor applies the features extracted through a convolutional neural network (CNN) model to a support vector data description (SVDD) technique to learn the distribution of normal and abnormal data, and determines abnormal data falling outside the normal range as declogging.

4. The apparatus of claim 1, wherein the processor collects the mold level and stopper position information and applies a cumulative sum control chart (CUSUM) model to determine the presence or absence of clogging.

5. The apparatus of claim 4, wherein the processor, based on the CUSUM model,
accumulates the change amount of the stopper position to detect real-time abnormal state,
calculates the change amount of the stopper position to evaluate the degree of blockage, and
analyzes the overall clogging progression state by checking whether the blockage state is accumulating.

6. The apparatus of claim 4, wherein the processor, based on the CUSUM model, comprehensively learns the features of gradual instability of the mold level, continuous rising pattern of the stopper position, and data showing outliers in the CUSUM indicators when clogging occurs, thereby detecting the presence or absence of clogging in real time.

7. A method for detecting abnormalities in a continuous casting process, the method comprising:
detecting a mold level and a stopper position in the continuous casting process; and
allowing a processor to learn information about the detected mold level and the stopper position and determine a mold level hunting occurrence type and a response method.

8. The method of claim 7 wherein the mold level and the stopper position is detected by a sensor module.

9. The method of claim 7, wherein in the determining of the mold level hunting occurrence type and the response method, the processor, for the purpose of the learning,
collects the mold level and stopper position information in real time,
analyzes time-series changes in the data using a convolutional neural network (CNN) model, and
extracts features that appear when declogging occurs.

10. The method of claim 7, wherein in the determining of the mold level hunting occurrence type and the response method, the processor applies the features extracted through a convolutional neural network (CNN) model to a support vector data description (SVDD) technique to learn the distribution of normal and abnormal data, and determines abnormal data falling outside the normal range as declogging.

11. The method of claim 7, wherein in the determining of the mold level hunting occurrence type and the response method, the processor collects the mold level and stopper position information and applies a cumulative sum control chart (CUSUM) model to determine the presence or absence of clogging.

12. The method of claim 10, wherein in the determining of the mold level hunting occurrence type and the response method, the processor, based on the CUSUM model,
accumulates the change amount of the stopper position to detect real-time abnormal state,
calculates the change amount of the stopper position to evaluate the degree of blockage, and
analyzes the overall clogging progression state by checking whether the blockage state is accumulating.

13. The method of claim 10, wherein in the determining of the mold level hunting occurrence type and the response method, the processor, based on the CUSUM model, comprehensively learns the features of gradual instability of the mold level, continuous rising pattern of the stopper position, and data showing outliers in the CUSUM indicators when clogging occurs, thereby detecting the presence or absence of clogging in real time.
